(19) United States
(12) Patent Application Publication
(10) Pub. No.: US 2008/0171774 A1
(43) Pub. Date: Jul. 17, 2008

(54) Nicotinanilides, Method for Production Thereof and Agents Comprising the Same for Prevention of Fungal Pests

(75) Inventors: Markus Gewehr, Kastellaun (DE); Jochen Dietz, Mannheim (DE); Thomas Grote, Wachenheim (DE); Carsten Blettner, Hong Kong (CN); Wassilios Grammenos, Ludwigshafen (DE); Udo Hunger, Mannheim (DE); Bernd Muller, Frankenthal (DE); Frank Schieweck, Hessheim (DE); Anja Schwogler, Mannheim (DE); Jan Klaas Lohmann, Mannheim (DE); Joachim Rheinheimer, Ludwigshafen (DE); Peter Schafer, Ottersheim (DE); Siegfried Strathmann, Limburgerhof (DE); Reinhard Stierl, Freinsheim (DE); Jan Rether, Kaiserslautern (DE); Karl Eicken, Wachenheim (DE)

Correspondence Address: BIRCH STEWART KOLASCH & BIRCH, PO BOX 747, FALLS CHURCH, VA, 22040-0747, US

(73) Assignee: BASF Akitengesellschaft, Ludwigshafen (DE)

(21) Appl. No.: 11/885,990

(22) PCT Filed: Mar. 15, 2006
(86) PCT No.: PCT/EP2006/060754

§ 371 Date: Sep. 10, 2007

(30) Foreign Application Priority Data
Mar. 16, 2005 (DE) 10 2005 012 485.2

(51) Int. Cl. C07D 213/63; C07D 213/56; A61K 31/4415; A61K 31/455

(52) U.S. Cl. 514/346; 514/355; 546/291; 546/316

(57) ABSTRACT

The present invention relates to nicotinanilides of the formula I

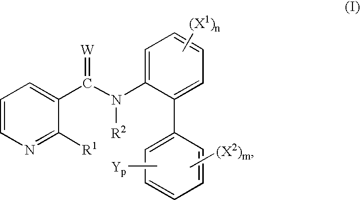

in which:
n=0, 1, 2;
m=2, 3;
X¹=F, chlorine;
X²=halogen;
Y=CN, NO₂, C₁-C₄-alkyl, C₁-C₄-haloalkyl, methoxy or methylthio;
p=0, 1;
R¹=halogen, methyl, C₁-C₄-haloalkyl, methoxy, methylthio, methylsulfinyl or methylsulfonyl;
R²=hydrogen, methyl or ethyl;
W=O, S;
   processes for preparing these compounds, compositions and seed comprising them and also methods for controlling harmful fungi.

The present invention relates to nicotinanilides of the formula I

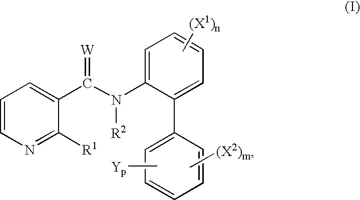

in which the variables are as defined below:
n is 0, 1 or 2;
m is 2 or 3;
X¹ is F or chlorine, where in the case of n=2 the two radicals X¹ may have different meanings;
X² is halogen, where the radicals X² may have different meanings;
Y is CN, NO₂, C₁-C₄-alkyl, C₁-C₄-haloalkyl, methoxy or methylthio;
p is zero or 1;
R¹ is halogen, methyl, C₁-C₄-haloalkyl, methoxy, methylthio, methylsulfinyl or methylsulfonyl;
R² is hydrogen, methyl or ethyl;
W is oxygen or sulfur.

Moreover, the invention relates to processes for preparing these compounds, to compositions comprising them and to methods for their use for controlling harmful fungi.

Nicotinanilides having fungicidal action are known from the literature. Thus, for example, EP-A 545 099 describes biphenylanilides of this type which are monosubstituted at the biphenyl group.

Nicotinanilides having a very specific substitution with unsaturated radicals at the nitrogen of the amide group are known from WO 02/059086.

2-Chloro-N-(4′-chloro-6-methylbiphenyl-2-yl)nicotinamide is known from JP-A 2001/302605.

It was an object of the present invention to provide nicotinanilides whose fungicidal action is better than that of the compounds of the prior art.

We have found that this object is achieved by the compounds I defined at the outset.

Moreover, we have found processes for preparing these compounds, compositions comprising them and methods for their use for controlling phytopathogenic harmful fungi.

Compared to the known compounds, the compounds of the formula I have improved activity against harmful fungi.

The compounds of the formula I may be present in various crystal modifications whose biological activities may differ. They are likewise provided by the present invention.

In the formula I, halogen is fluorine, chlorine, bromine or iodine, preferably fluorine or chlorine.
C₁-C₄-alkyl is methyl, ethyl, n-propyl, 1-methylethyl, n-butyl, 1-methylpropyl, 2-methylpropyl or 1,1-dimethylethyl, preferably methyl or ethyl;
C₁-C₄-haloalkyl is a partially or fully halogenated C₁-C₄-alkyl radical, the halogen atom(s) being in particular fluorine and/or chlorine, i.e., for example, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2-chloro-2-fluoroethyl, 2,2,2-trifluoroethyl, 2-chloro-1,1,2-trifluoroethyl, 2-chloro-2,2-difluoroethyl, 2-bromo-2,2-difluoroethyl, 2,2-dichloro-2-fluoroethyl, 2,2,2-trichloroethyl, 1,1,2,2-tetrafluoroethyl, 1,1,2,2-tetrachloroethyl, pentafluoroethyl, 2,2,3,3-tetrafluoro-1-propyl, 1,1,2,3,3,3-hexafluoro-1-propyl, 1,1,1,3,3,3-hexafluoro-2-propyl, heptafluoro-1-propyl, heptafluoro-2-propyl, 2,2,3,3,4,4,4-heptafluoro-1-butyl or nonafluoro-1-butyl, in particular halomethyl, particularly preferably CH₂Cl. CH(Cl)₂, CH₂—F, CH(F)₂, CF₃, CHFC₁, CF₂Cl or CF(Cl)₂.

The compounds I are generally obtained by reacting a carbonyl halide of the formula II in a manner known per se (for example J. March, Advanced Organic Chemistry, 2nd Ed., 382 f., McGraw-Hill, 1977) in the presence of a base with an aniline of the formula III:

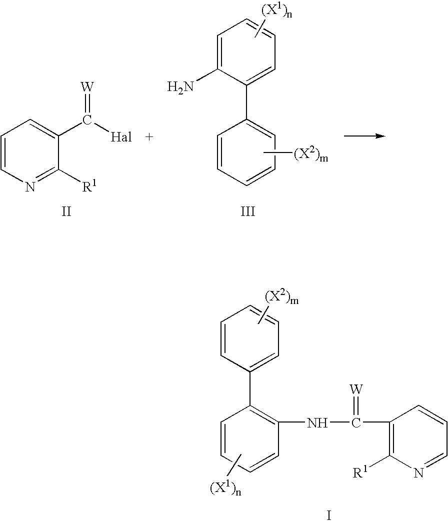

In the formula II, the radical Hal denotes a halogen atom, such as fluorine, chlorine, bromine and iodine, in particular fluorine, chlorine or bromine. This reaction is usually carried out at temperatures of from (−20)° C. to 100° C., preferably from 0° C. to 50° C.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also methylene chloride, dimethyl sulfoxide and dimethylformamide, particularly preferably toluene, methylene chloride and tetrahydrofuran.

It is also possible to use mixtures of the solvents mentioned.

Suitable bases are, in general, inorganic compounds, for example alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate and calcium carbonate, and also alkali metal bicarbonates, such as sodium bicarbonate, and organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines.

Particular preference is given to using triethylamine and pyridine.

The bases are generally employed in equimolar amounts, based on the compound II. However, they can also be used in an excess of from 5 mol % to 30 mol %, preferably from 5 mol % to 10 mol %, or—if tertiary amines are used—, if appropriate, as solvents.

The starting materials are generally reacted with one another in approximately equimolar amounts. In terms of yield, it may be advantageous to employ II in an excess of from 1 mol % to 20 mol %, preferably from 1 mol % to 10 mol %, based on III.

The starting materials of the formulae II or III required for preparing the compounds I are known or can be synthesized analogously to known compounds (Helv. Chim. Acta, 60, 978 (1977); Zh. Org. Khim., 26, 1527 (1990); Heterocycles 26, 1885 (1987); Izv. Akad. Nauk. SSSR Ser. Khim., 2160 (1982); THL 28, 593 (1987); THL 29, 5463 (1988)).

Furthermore, it has been found that compounds of the formula I are obtained by reacting, in a known manner, carboxylic acids of the formula IV with an aniline of the formula III in the presence of dehydrating agents and, if appropriate, an organic base.

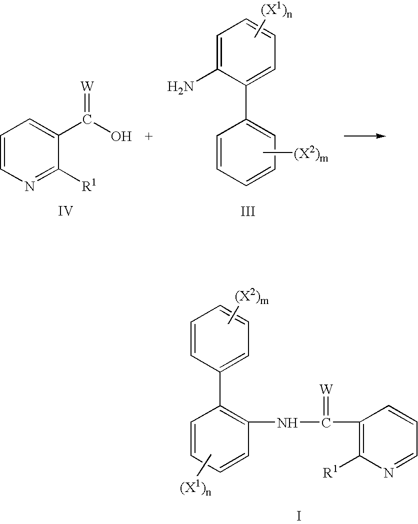

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, and also dimethyl sulfoxide and dimethylformamide, particularly preferably methylene chloride, toluene and tetrahydrofuran.

It is also possible to use mixtures of the solvents mentioned.

Suitable dehydrating agents are 1,1′-carbonyldiimidazole, bis(2-oxo-3-oxazolidinyl)-phosphoryl chloride, carbodiimides, such as N,N′-dicyclohexylcarbodiimide and N-(3-dimethylaminopropyl)-N′-ethylcarbodiimide, phosphonium salts, such as (benzotriazol-1-yloxy)tris(dimethylamino)phosphonium hexafluorophosphate, bromotripyrrolidinophosphonium hexafluorophosphate, bromotris(dimethyl-amino)phosphonium hexafluorophosphate and chlorotripyrrolidinophosphonium-hexafluorophosphate, uronium and thiuronium salts, such as O-(benzotriazol-1-yl)-N,N,N′,N′-tetramethyluronium hexafluorophosphate, O-(7-azabenzotriazol-1-yl)-N,N,N′,N′-tetramethyluronium hexafluorophosphate, S-(1-oxido-2-pyridyl)-N,N,N′,N′-tetramethylthiuronium tetrafluoroborate, O-(2-oxo-1(2H)pyridyl)-N,N,N′,N′-tetramethyluronium tetrafluoroborate and O-[(ethoxycarbonyl)cyanomethylenamino]-N,N,N′,N′-tetramethyluronium tetrafluoroborate, carbenium salts, such as (benzotriazol-1-yloxy)dipyrrolidinocarbenium hexafluorophosphate, (benzotriazol-1-yloxy)dipiperidinocarbenium hexafluorophosphate, O-(3,4-dihydro-4-oxo-1,2,3-benzotriazin-3-yl)-N,N,N′,N′-tetramethyluronium tetrafluoroborate, chloro-N′,N′-bis(tetramethylene)formamidinium tetrafluoroborate, chlorodipyrrolidinocarbenium hexafluorophosphate, chloro-N,N,N′,N′-bis(pentamethylene)formamidinium tetrafluoroborate, imidazolium salts, such as 2-chloro-1,3-dimethylimidazolidinium tetrafluoroborate, preferably 1,1′-carbonyldiimidazole, bis(2-oxo-3-oxazolidinyl)phosphoryl chloride, N,N′-dicyclohexylcarbodiimide and N-(3-dimethyl-aminopropyl)-N′-ethylcarbodiimide.

Suitable organic bases are tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to using triethylamine and pyridine. The bases are generally employed in an excess of from 10 mol % to 200 mol %, preferably from 50 mol % to 150 mol %, based on the compound IV.

The starting materials are generally reacted with one another in approximately equimolar amounts. In terms of yield, it may be advantageous to use an excess of from 1 mol % to 20 mol %, preferably from 1 mol % to 10 mol %, of one of the compounds. The dehydrating agents are generally employed in an excess of from 5 mol % to 100 mol %, preferably from 5 mol % to 60 mol %.

The starting materials of the formulae III and IV required for preparing the compounds I are known or can be synthesized analogously to known compounds.

Compounds of the formula I where R²=methyl or ethyl are obtained by reacting compounds of the formula I where R²H in a known manner in the presence of a base with an alkylating agent.

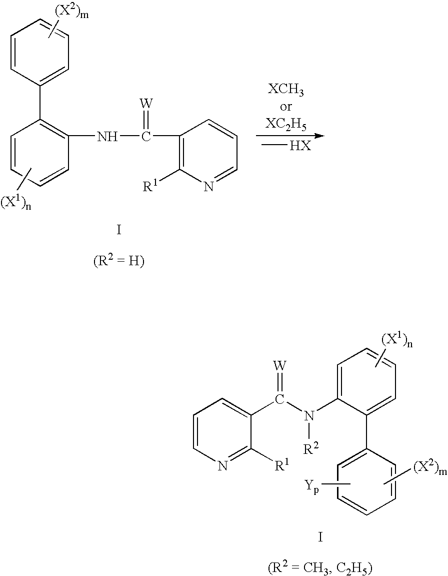

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, and also dimethyl sulfoxide and dimethyl-formamide, particularly preferably diethyl ether, tert-butyl methyl ether, tetrahydrofuran and dimethylformamide.

It is also possible to use mixtures of the solvents mentioned.

Suitable alkylating agents (XCH₃ or XC₂H₅) are alkyl halides, such as methyl iodide, ethyl iodide, methyl bromide, ethyl bromide, methyl chloride and ethyl chloride, alkyl perfluoroalkylsulfonates, such as methyl trifluoromethylsulfonate and ethyl trifluoromethylsulfonate, alkyl alkylsulfonates, such as methyl methylsulfonate and ethyl methylsulfonate, alkyl arylsulfonates, such as methyl p-tolylsulfonate and ethyl p-tolylsulfonate, oxonium salts, such as trimethyloxonium tetrafluoroborate and triethyloxonium tetrafluoroborate.

Particular preference is given to methyl iodide, ethyl iodide, methyl bromide, ethyl bromide, methyl chloride and ethyl chloride.

Suitable bases are, in general, inorganic compounds, for example alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, sodium carbonate, potassium carbonate and calcium carbonate, and also alkali metal bicarbonates, such as sodium bicarbonate, or organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide.

Particular preference is given to using sodium carbonate, potassium carbonate, sodium hydride, potassium hydride, butyllithium and potassium tert-butoxide.

The bases are generally employed in approximately equimolar amounts, based on the compound I. However, they can also be used in an excess of from 5 mol % to 30 mol %, preferably from 5 mol % to 10 mol %.

The starting materials are generally reacted with one another in approximately equimolar amounts. However, in terms of yield, it may also be advantageous to employ the alkylating agent in an excess of from 1 mol % to 20 mol %, preferably from 1 mol % to 10 mol %, based on 1.

The compounds I in which W is sulfur can also be prepared by sulfurization of the corresponding compounds I in which W is oxygen (cf., for example, D. Petrova & K. Jakobcic, Croat. Chem. Acta 48, 49 (1976) and also WO 01/42223).

With a view to the biological activity of the compounds I, preference is given to the following meanings of the variables, in each case on their own or in combination:
n is zero;
X¹ is chlorine;
X² is F or Cl, in particular fluorine;
Y is C₁-C₄-alkyl, C₁-C₄-haloalkyl or methoxy, in particular methyl, difluoromethyl, trifluoromethyl or methoxy;
   particularly preferably methyl or trifluoromethyl;
p is zero;
R¹ is halogen, methyl, trifluoromethyl or methoxy, in particular F, Cl, methyl or trifluoromethyl,
   particularly preferably fluorine, chlorine or trifluoromethyl, in particular chlorine or trifluoromethyl, very particularly preferably chlorine;
R² is hydrogen or methyl, in particular hydrogen;
W is oxygen.

In the case of m=3, the radicals X² are preferably located in the 2,4,5- or 3,4,5-position, in particular in the 3,4,5-position.

Particular preference is given to compounds I having the following combinations of substituents, where the substituents are as defined below:
X² is F or chlorine;
Y is C₁-C₄-alkyl, C₁-C₄-haloalkyl or methoxy, in particular methyl, difluoromethyl, trifluoromethyl or methoxy;
R¹ is halogen, methyl, trifluoromethyl or methoxy, in particular fluorine, chlorine, methyl or trifluoromethyl;
R² is hydrogen or methyl;
W is oxygen.

Preference is furthermore also given to the following combinations of substituents having the following meanings:
n is zero;
p is zero;
X² is F or chlorine;
R¹ is F, Cl, methyl or trifluoromethyl, in particular fluorine or chlorine;
R² is hydrogen;
W is oxygen.

Preference is also given to compounds I where m=2, in particular to those compounds in which n is zero, p is zero, R¹ is fluorine or chlorine, in particular chlorine, and R² is hydrogen.

Here, the radicals X are preferably located in the 2,4- or 3,4-position, in particular in the 3,4-position.

With a view to their use as fungicides, preference is given to the compounds of the formulae I-A and I-B.

TABLE A

| | |
|---|---|
| | (I-A) |
| 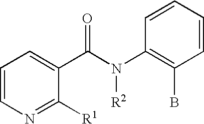 | |
| | (I-B) |
| 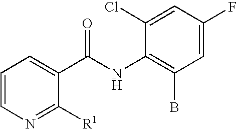 | |

| No. | B |
|---|---|
| 1 | 2,3-dichlorophenyl |
| 2 | 2,4-dichlorophenyl |
| 3 | 2,5-dichlorophenyl |
| 4 | 2,6-dichlorophenyl |
| 5 | 3,4-dichlorophenyl |
| 6 | 3,5-dichlorophenyl |
| 7 | 2,3-difluorophenyl |
| 8 | 2,4-difluorophenyl |
| 9 | 2,5-difluorophenyl |
| 10 | 2,6-difluorophenyl |
| 11 | 3,4-difluorophenyl |
| 12 | 3,5-difluorophenyl |
| 13 | 2-chloro-3-fluorophenyl |
| 14 | 2-chloro-4-fluorophenyl |
| 15 | 2-chloro-5-fluorophenyl |
| 16 | 2-chloro-6-fluorophenyl |
| 17 | 3-chloro-2-fluorophenyl |
| 18 | 3-chloro-4-fluorophenyl |
| 19 | 3-chloro-5-fluorophenyl |
| 20 | 3-chloro-6-fluorophenyl |
| 21 | 4-chloro-2-fluorophenyl |
| 22 | 4-chloro-3-fluorophenyl |
| 23 | 2,3,4-trichlorophenyl |
| 24 | 2,3,5-trichlorophenyl |
| 25 | 2,3,6-trichlorophenyl |
| 26 | 2,4,5-trichlorophenyl |
| 27 | 2,4,6-trichlorophenyl |
| 28 | 3,4,5-trichlorophenyl |
| 29 | 2,3,4-trifluorophenyl |
| 30 | 2,3,5-trifluorophenyl |
| 31 | 2,3,6-trifluorophenyl |
| 32 | 2,4,5-trifluorophenyl |
| 33 | 2,4,6-trifluorophenyl |
| 34 | 3,4,5-trifluorophenyl |
| 35 | 2-chloro-3,4-difluorophenyl |
| 36 | 2-chloro-4,5-difluorophenyl |
| 37 | 2-chloro-5,6-difluorophenyl |
| 38 | 2-chloro-3,5-difluorophenyl |
| 39 | 2-chloro-3,6-difluorophenyl |
| 40 | 2-chloro-4,6-difluorophenyl |
| 41 | 3-chloro-2,4-difluorophenyl |
| 42 | 3-chloro-2,5-difluorophenyl |
| 43 | 3-chloro-2,6-difluorophenyl |
| 44 | 3-chloro-4,5-difluorophenyl |
| 45 | 3-chloro-4,6-difluorophenyl |
| 46 | 3-chloro-5,6-difluorophenyl |
| 47 | 4-chloro-2,3-difluorophenyl |
| 48 | 4-chloro-2,5-difluorophenyl |
| 49 | 4-chloro-2,6-difluorophenyl |
| 50 | 4-chloro-3,5-difluorophenyl |
| 51 | 2-fluoro-3,4-dichlorophenyl |
| 52 | 2-fluoro-4,5-dichlorophenyl |
| 53 | 2-fluoro-5,6-dichlorophenyl |
| 54 | 2-fluoro-3,5-dichlorophenyl |
| 55 | 2-fluoro-3,6-dichlorophenyl |
| 56 | 2-fluoro-4,6-dichlorophenyl |
| 57 | 3-fluoro-2,4-dichlorophenyl |
| 58 | 3-fluoro-2,5-dichlorophenyl |
| 59 | 3-fluoro-2,6-dichlorophenyl |
| 60 | 3-fluoro-4,5-dichlorophenyl |
| 61 | 3-fluoro-4,6-dichlorophenyl |
| 62 | 3-fluoro-5,6-dichlorophenyl |
| 63 | 4-fluoro-2,3-dichlorophenyl |
| 64 | 4-fluoro-2,5-dichlorophenyl |
| 65 | 4-fluoro-2,6-dichlorophenyl |
| 66 | 4-fluoro-3,5-dichlorophenyl |

### Table 1:

Compounds of the formula I-A in which R¹ is C¹ and R² is hydrogen and B corresponds in each case to one row of Table A.

### Table 2:

Compounds of the formula I-A in which R¹ is F and R² is hydrogen and B corresponds in each case to one row of Table A.

### Table 3:

Compounds of the formula I-A in which R¹ is methyl and R² is hydrogen and B corresponds in each case to one row of Table A.

### Table 4:

Compounds of the formula I-A in which R¹ is trifluoromethyl and R² is hydrogen and B corresponds in each case to one row of Table A.

### Table 5:

Compounds of the formula I-A in which R¹ is methoxy and R² is hydrogen and B corresponds in each case to one row of Table A.

### Table 6:

Compounds of the formula I-A in which R¹ is C¹ and R² is methyl and B corresponds in each case to one row of Table A.

### Table 7:

Compounds of the formula I-A in which R¹ is F and R² is methyl and B corresponds in each case to one row of Table A.

### Table 8:

Compounds of the formula I-A in which R¹ is methyl and R² is methyl and B corresponds in each case to one row of Table A.

### Table 9:

Compounds of the formula I-A in which R¹ is trifluoromethyl and R² is methyl and B corresponds in each case to one row of Table A.

### Table 10:

Compounds of the formula I-A in which R¹ is methoxy and R² is methyl and B corresponds in each case to one row of Table A.

### Table 11:

Compounds of the formula I-A in which R¹ is C¹ and R² is ethyl and B corresponds in each case to one row of Table A.

### Table 12:

Compounds of the formula I-A in which R¹ is F and R² is ethyl and B corresponds in each case to one row of Table A.

### Table 13:

Compounds of the formula I-A in which R¹ is methyl and R² is ethyl and B corresponds in each case to one row of Table A.

### Table 14:

Compounds of the formula I-A in which R¹ is trifluoromethyl and R² is ethyl and B corresponds in each case to one row of Table A.

### Table 15:

Compounds of the formula I-A in which R¹ is methoxy and R² is ethyl and B corresponds in each case to one row of Table A.

### Table 16:

Compounds of the formula I-B in which R¹ is Cl and B corresponds in each case to one row of Table A.

### Table 17:

Compounds of the formula I-B in which R¹ is F and B corresponds in each case to one row of Table A.

### Table 18:

Compounds of the formula I-B in which R¹ is methyl and B corresponds in each case to one row of Table A.

### Table 19:

Compounds of the formula I-B in which R¹ is trifluoromethyl and B corresponds in each case to one row of Table A.

### Table 20:

Compounds of the formula I-B in which R¹ is methoxy and B corresponds in each case to one row of Table A.

Very particular preference is given to the following nicotinanilides of the formula I:
2-chloro-N-(3′,4′-dichlorobiphenyl-2-yl)nicotinamide,
2-chloro-N-(3′,5′-dichlorobiphenyl-2-yl)nicotinamide,
2-chloro-N-(3′-chloro-4′-fluorobiphenyl-2-yl)nicotinamide,
2-chloro-N-(4′-chloro-3′-fluorobiphenyl-2-yl)nicotinamide,
2-chloro-N-(3′,4′-difluorobiphenyl-2-yl)nicotinamide,
2-chloro-N-(2′,4′-dichlorobiphenyl-2-yl)nicotinamide,
2-chloro-N-(2′,5′-difluorobiphenyl-2-yl)nicotinamide,
2-chloro-N-(4′-chloro-2′-fluorobiphenyl-2-yl)nicotinamide,
2-chloro-N-(2′,4′-difluorobiphenyl-2-yl)nicotinamide,
2-chloro-N-(4-fluoro-2′,4′-dichlorobiphenyl-2-yl)nicotinamide,
2-chloro-N-(3′,4′,5′-trifluorobiphenyl-2-yl)nicotinamide,
2-chloro-N-(2′,4′,5′-trifluorobiphenyl-2-yl)nicotinamide,
2-chloro-N-(4′-chloro-2′-fluoro-5′-methoxybiphenyl-2-yl)nicotinamide,
2-trifluoromethyl-N-(3′,4′-dichlorobiphenyl-2-yl)nicotinamide,
2-trifluoromethyl-N-(4-fluoro-2′,4′-dichlorobiphenyl-2-yl)nicotinamide,
2-trifluoromethyl-N-(3′,4′,5′-trifluorobiphenyl-2-yl)nicotinamide,
2-trifluoromethyl-N-(2′,4′,5′-trifluorobiphenyl-2-yl)nicotinamide,
2-trifluoromethyl-N-(4′-chloro-2′-fluoro-5′-methoxybiphenyl-2-yl)nicotinamide,
2-trifluoromethyl-N-(3′,4′-difluorobiphenyl-2-yl)nicotinamide,
2-trifluoromethyl-N-(3′,5′-dichlorobiphenyl-2-yl)nicotinamide,
2-trifluoromethyl-N-(2′,4′-dichlorobiphenyl-2-yl)nicotinamide,
2-trifluoromethyl-N-(3′-chloro-4′-fluorobiphenyl-2-yl)nicotinamide,
2-chloro-N-(3′,4′,5′-trifluorobiphenyl-2-yl)-N-methylnicotinamide and 2-trifluoromethyl-N-(3′,4′,5′-trifluorobiphenyl-2-yl)-N-ethylnicotinamide.

The compounds I are suitable as fungicides. They are distinguished by an outstanding effectiveness against a broad spectrum of phytopathogenic fungi, especially from the classes of the Ascomycetes, Deuteromycetes, Peronosporomycetes (syn. Oomycetes) and Basidiomycetes. Some are systemically effective and they can be used in plant protection as foliar fungicides, soil fungicides and fungicides.

They are particularly important in the control of a multitude of fungi on various cultivated plants, such as wheat, rye, barley, oats, rice, corn, lawn, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns; for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, for example Cochliobolus sativus on cereals, Cochliobolus miyabeanus on rice;
Colletotricum species on soybeans and cotton;
Drechslera species, Pyrenophora species on corn, cereals, rice and lawns, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumbers;
Fusarium and Verticillium species on various plants, for example, F. graminearum or F. culmorum on cereals or F. oxysporum on a multitude of plants, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and peanuts, for example, M. graminicola on wheat or M. fijiesis on bananas;
Peronospora species on cabbage and bulbous plants, for example, P. brassicae on cabbage or P. destructor on onions;
Phakopsara pachyrhizi and Phakopsara meibomiae on soybeans;
Phomopsis species on soybeans and sunflowers;
Phytophthora infestans on potatoes and tomatoes;
Phytophthora species on various plants, for example, P. capsici on bell pepper;
Plasmopara viticola on grapevines;
Podosphaera leucotricha on apples;
Pseudocercosporella herpotrichoides on cereals;
Pseudoperonospora on various plants, for example, P. cubensis on cucumber or P. humili on hops;
Puccinia species on various plants, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, for example, P. ultiumum on various plants, P. aphanidermatum on lawns;
Rhizoctonia species on cotton, rice, potatoes, lawns, corn, oilseed rape, sugar beet, vegetables and on various plants, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, for example, U. maydis on corn;
Venturia species (scab) on apples and pears, for example, V. inaequalis on apples.

The compositions according to the invention are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95% by weight, preferably between 0.5 and 90% by weight, of active compound.

When employed in plant protection, the amounts applied are, depending on the kind of effect desired, between 0.01 and 2.0 kg of active compound per ha.

In seed treatment, for example by dusting, coating or drenching, amounts of active compound of 1 to 1000 g/100 kg, preferably 5 to 100 g/100 kg of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso® products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone and NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalene-sulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

Formulations for seed treatment may further comprise binders and/or gelants and if appropriate dyes.

Binders can be added to increase the adhesion of the active compounds to the seed after the treatment. Suitable binders are for example EO/PO block copolymer surfactants, but also polyvinyl alcohols, polyvinylpyrrolidones, polyacrylates, polymethacrylates, polybutenes, polyisobutylenes, polystyrenes, polyethyleneamines, polyethyleneamides, polyethyleneimines (Lupasol®, Polymin®), polyethers, polyurethanes, polyvinyl acetates, Tylose and copolymers of these polymers. A suitable gelant is for example carrageen (Satiagel®)).

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The concentrations of active compound in the ready-to-use preparations can be varied within relatively wide ranges. In general, they are between 0.0001 and 10%, preferably between 0.01 and 1%.

The active compounds can also be used with great success in the ultra-low-volume (ULV) process, it being possible to apply formulations with more than 95% by weight of active compound or even the active compound without additives.

For the treatment of seed, the formulations in question give, after two-to-tenfold dilution, active compound concentrations of from 0.01 to 60% by weight, preferably from 0.1 to 40% by weight, in the ready-to-use preparations.

The following are examples of formulations: 1. Products for dilution with water

### A) Water-Soluble Concentrates (SL)

10 parts by weight of a compound I according to the invention are dissolved with 90 parts by weight of water or with a water-soluble solvent. As an alternative, wetters or other auxiliaries are added. The active compound dissolves upon dilution with water. This gives a formulation having an active compound content of 10% by weight.

### B) Dispersible Concentrates (DC)

20 parts by weight of a compound I according to the invention are dissolved in 70 parts by weight of cyclohexanone with addition of 10 parts by weight of a dispersant, for example polyvinylpyrrolidone. Dilution with water gives a dispersion. The active compound content is 20% by weight

### C) Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D) Emulsions (EW, EO)

25 parts by weight of a compound I according to the invention are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E) Suspensions (SC, OD)

In an agitated ball mill, 20 parts by weight of a compound I according to the invention are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F) Water-Dispersible Granules and Water-Soluble Granules (WG, SG)

50 parts by weight of a compound I according to the invention are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G) Water-Dispersible Powders and Water-Soluble Powders (WP, SP)

75 parts by weight of a compound I according to the invention are ground in a rotor-stator mill with addition of 25 parts by weight of dispersants, wetters and silica gel. Dilution with water gives a stable dispersion or solution of the active compound. The active compound content of the formulation is 75% by weight.

### 2. Products to be Applied Undiluted

### H) Dustable Powders (DP)

5 parts by weight of a compound I according to the invention are ground finely and mixed intimately with 95 parts by weight of finely divided kaolin. This gives a dustable product with an active compound content of 5% by weight.

### J) Granules (GR, FG, GG, MG)

0.5 part by weight of a compound I according to the invention is ground finely and associated with 99.5 parts by weight of carriers. Current methods are extrusion, spray-drying or the fluidized bed. This gives granules with an active compound content of 0.5% by weight to be applied undiluted.

### K) ULV Solutions (UL)

10 parts by weight of a compound I according to the invention are dissolved in 90 parts by weight of an organic solvent, for example xylene. This gives a product with an active compound content of 10% by weight to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents are usually admixed with the agents according to the invention in a weight ratio of 1:100 to 100:1, preferably of 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30%; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the application form as fungicides, also be present together with other active compounds, e.g. with herbicides, insecticides, growth regulators such as prohexadione-Ca, fungicides or also with fertilizers. Mixing the compounds I or the compositions comprising them with other active compounds, in particular fungicides, it is in many cases possible, for example, to broaden the activity spectrum or to prevent the development of resistance. In many cases, synergistic effects are obtained.

The following list of fungicides, with which the compounds according to the invention can be used in conjunction, is intended to illustrate the possible combinations but does not limit them:

strobilurins
azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;
   carboxamides
   carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoro-methyl-2-methylthiazole-5-carboxamide, N-(4′-chloro-3′-fluorobiphenyl-2-yl)-4-di-fluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
   carboxylic acid morpholides: dimethomorph, flumorph;
   benzamides: flumetover, fluopicolide (picobenzamid), zoxamide;
   other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;
      azoles
   triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
   imidazoles: cyazofamid, imazalil, pefurazoate, prochloraz, triflumizole;
   benzimidazoles: benomyl, carbendazim, fuberidazole, thiabendazole;
   others: ethaboxam, etridiazole, hymexazole;
      nitrogenous heterocyclyl compounds
   pyridines: fluazinam, pyrifenox, 3-[5-(4-chlorophenyl)-2,3-dimethylisoxazolidin-3-yl]-pyridine;
   pyrimidines: bupirimate, cyprodinil, ferimzone, fenarimol, mepanipyrim, nuarimol,
   pyrimethanil;
   piperazines: triforine;
   pyrroles: fludioxonil, fenpiclonil;
   morpholines: aldimorph, dodemorph, fenpropimorph, tridemorph;
   dicarboximides: iprodione, procymidone, vinclozolin;
   others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;
      carbamates and dithiocarbamates
   dithiocarbamates: ferbam, mancozeb, maneb, metiram, metam, propineb, thiram, zineb, ziram;
   carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
      other fungicides
   guanidines: dodine, iminoctadine, guazatine;
   antibiotics: kasugamycin, polyoxins, streptomycin, validamycin A;
   organometallic compounds: fentin salts;
   sulfur-containing heterocyclyl compounds: isoprothiolane, dithianon;
   organophosphorus compounds: edifenphos, fosetyl, fosetyl-aluminum, iprobenfos, pyrazophos, tolclofos-methyl, phosphorous acid and its salts;
   organochlorine compounds: thiophanate-methyl, chlorothalonil, dichlofluanid, tolylfluanid, flusulfamide, phthalide, hexachlorobenzene, pencycuron, quintozene;
   nitrophenyl derivatives: binapacryl, dinocap, dinobuton;
   inorganic active compounds: Bordeaux mixture, copper acetate, copper hydroxide, copper oxychloride, basic copper sulfate, sulfur;
   others: spiroxamine, cyflufenamid, cymoxanil, metrafenone.

### SYNTHESIS EXAMPLES

### Example 1

### N-(3′,4′-Dichlorobiphenyl-2-yl)-2-(trifluoromethyl)nicotinamide

At 0° C., 0.25 g of 3′,4′-dichloro-2-aminobiphenyl and 0.40 g of bis-(2-oxo-3-oxazolidinyl)phosphoryl chloride were added to a solution of 0.20 g of 2-(trifluoromethyl)nicotinic acid and 0.21 g of triethylamine in 30 ml dichloromethane. The mixture was stirred at 0° C. for 15 minutes and then at room temperature for 12 hours. The mixture was then washed successively with dilute hydrochloric acid, twice with aqueous sodium bicarbonate solution and with water. The organic phase was dried and concentrated. The crude product was purified by column chromatography on silica gel using cyclohexane/methyl tert-butyl ether 1:2. This gave 0.21 g of the desired product as beige crystals.

### 2-Chloro-N-(4′-chloro-2′-fluorobiphenyl-2-yl)nicotinamide

At room temperature, 0.23 g of 2-chloronicotinoyl chloride were added dropwise to a solution of 0.29 g of 4′-chloro-2′-fluoro-2-aminobiphenyl and 0.15 g of pyridine in 10 ml of toluene, and the mixture was stirred at room temperature for another 16 hours. 10 ml of tetrahydrofuran and 30 ml of methyl tert-butyl ether were added, and the organic phase was washed successively with 2% strength hydrochloric acid, 2% strength aqueous sodium hydroxide solution and then with dilute aqueous sodium chloride solution. The organic phase was dried and concentrated under reduced pressure. The crude product was triturated with 5 ml of diisopropyl ether and the solid that remained was separated off with suction and dried. This gave 0.34 g of the desired product as a white powder of m.p. 124-126° C.

The compounds of the formula I where W═O listed in Table 21 below were prepared according to the illustrated methods.

TABLE 21

| | | | | | | | | | Characterization |
|---|---|---|---|---|---|---|---|---|---|
| Example | R¹ | R² | X₁ | n | X₂ | m | Y | p | (m.p. or¹H NMR) |
| 21.1 | Cl | H | — | 0 | 3,4-Cl₂ | 2 | — | 0 | 112-118° C. |
| 21.2 | Cl | H | — | 0 | 3,5-Cl₂ | 2 | — | 0 | 147-149° C. |
| 21.3 | Cl | H | — | 0 | 4-F,3-Cl | 2 | — | 0 | 144-145° C. |
| 21.4 | Cl | H | — | 0 | 3-F,4-Cl | 2 | — | 0 | 113-115° C. |
| 21.5 | Cl | H | — | 0 | 3,4-F₂ | 2 | — | 0 | 123-125° C. |
| 21.6 | Cl | H | — | 0 | 2-Cl, 4-Cl | 2 | — | 0 | 105-109° C. |
| 21.7 | CF₃ | H | — | 0 | 3,4-Cl₂ | 2 | — | 0 | 150-153° C. |
| 21.8 | Cl | H | — | 0 | 2,5-F₂ | 2 | — | 0 | 74-75° C. |
| 21.9 | Cl | H | — | 0 | 2-F, 4-Cl | 2 | — | 0 | 124-126° C. |
| 21.10 | Cl | H | — | 0 | 2,4-F₂ | 2 | — | 0 | 117-118° C. |
| 21.11 | Cl | H | 4-F | 1 | 2,4-Cl₂ | 2 | — | 0 | 150-155° C. |
| 21.12 | Cl | H | — | 0 | 3,4,5-F₃ | 3 | — | 0 | 139-144° C. |
| 21.13 | Cl | H | — | 0 | 2,4,5-F₃ | 3 | — | 0 | 103-110° C. |
| 21.14 | Cl | H | — | 0 | 2-F, 4-Cl | 2 | 5-OCH₃ | 1 | 159-162° C. |
| 21.15 | CF₃ | H | 4-F | 1 | 2,4-Cl₂ | 2 | — | 0 | 164-169° C. |
| 21.16 | CF₃ | H | — | 0 | 3,4,5-F₃ | 3 | — | 0 | 164-168° C. |
| 21.17 | CF₃ | H | — | 0 | 2,4,5-F₃ | 3 | — | 0 | 141-144° C. |
| 21.18 | CF₃ | H | — | 0 | 2-F,4-Cl | 2 | 5-OCH₃ | 1 | 151-156° C. |
| 21.19 | CF₃ | H | — | 0 | 3,4-F₂ | 2 | — | 0 | 121-125° C. |
| 21.20 | CF₃ | H | — | 0 | 3,5-Cl₂ | 2 | — | 0 | 161-165° C. |
| 21.21 | CF₃ | H | — | 0 | 2,4-Cl₂ | 2 | — | 0 | 143-147° C. |
| 21.22 | CF₃ | H | — | 0 | 3-Cl, 4-F | 2 | — | 0 | 167-170° C. |
| 21.23 | Cl | CH₃ | | 0 | 3,4,5-F₃ | 3 | — | 0 | ¹H-NMR (CDCl₃): δ = |
| | | | | | | | | | 8.50-8.20 (m, 1H), |
| | | | | | | | | | 7.60-7.10 (m, 6H), |
| | | | | | | | | | 6.95-6.60 (m, 2H), |
| | | | | | | | | | 3.55 (s, 3H) |
| 21.24 | CF₃ | CH₂CH₃ | — | 0 | 3,4,5-F₃ | 3 | — | 0 | 94-99° C. |

### Examples of the Action Against Harmful Fungi

The fungicidal activity of the compounds of the formula I was demonstrated by the following tests:

### Use Examples

The active compounds were prepared as a stock solution comprising 25 mg of active compound which was made up to 10 ml using a mixture of acetone and/or dimethyl sulfoxide and the emulsifier Uniperol® EL (wetting agent having an emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the desired concentration of active compound.

### Use Example 1

### Activity Against Early Blight of Tomato Caused by Altemaria solani

Leaves of potted plants of the cultivar “Goldene Königin” were sprayed to runoff point with an aqueous suspension having the concentration of active compound stated below. The next day, the leaves were infected with an aqueous spore suspension of Altemaria solani in 2% biomalt solution having a density of 0.17×10⁶ spores/ml. The plants were then placed in a water-vapor-saturated chamber at temperatures of between 20 and 22° C. After 5 days, the infection on the untreated, but infected control plants had developed to such an extent that the infection could be determined visually in %.

In this test, the plants which had been treated with 250 mg/l of compounds 21.1, 21.2, 21.3, 21.4, 21.5 and 21.7 from Table 21 showed an infection of at most 20%, whereas the untreated plants were 64% infected.

### Use Example 2

### Activity Against Net Blotch of Barley Caused by Pyrenophora teres, 1 Day Protective Application

Leaves of potted barley seedlings of the cultivar “Hanna” were sprayed to runoff point with an aqueous suspension having the concentration of active compound stated below. 24 h after the spray coating had dried on, the test plants were inoculated with an aqueous spore suspension of Pyrenophora [syn. Drechslera] teres, the net blotch pathogen. The test plants were then placed in a greenhouse at temperatures of from 20 to 24° C. and 95-100% relative atmospheric humidity. After 6 days, the extent of the development of the fungal infection on the leaves was determined visually in % of the entire leaf surface.

In this test, the plants which had been treated with 250 mg/l of compounds 21.1, 21.2, 21.3, 21.4, 21.5 and 21.7 from Table 21 showed an infection of at most 15%, whereas the untreated plants were 80% infected.

1. A nicotinanilide of the formula I 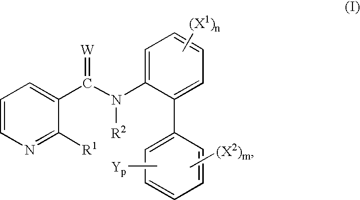
in which the variables are as defined below:
n is 0, 1 or 2;
m is 2 or 3;
X¹ is F or chlorine, where in the case of n=2 the two radicals X¹ may have different meanings;
X² is halogen, where the radicals X² may have different meanings;
Y is CN, NO₂, C₁-C₄-alkyl, C₁-C₄-haloalkyl, methoxy or methylthio;
p is zero or 1;
R¹ is halogen, methyl, C₁-C₄-haloalkyl, methoxy, methylthio, methylsulfinyl or methylsulfonyl;
R² is hydrogen, methyl or ethyl;
W is oxygen or sulfur.

2. The nicotinanilide of the formula I according to claim 1 in which the variables are as defined below:
X² is F or chlorine;
Y is C₁-C₄-alkyl, C₁-C₄-haloalkyl or methoxy;
R¹ is halogen, methyl, trifluoromethyl or methoxy;
R² is hydrogen or methyl;
W is oxygen.

3. The nicotinanilide of the formula I according to claim 1 in which the variables are as defined below:
X² is F or chlorine;
Y is methyl, difluoromethyl, trifluoromethyl or methoxy;
R¹ is F, Cl, methyl or trifluoromethyl;
R² is hydrogen or methyl;
W is oxygen.

4. The nicotinanilide of the formula I according to claim 1 in which the variables are as defined below:
n is zero;
p is zero;
X² is F or chlorine;
R¹ is F, Cl, methyl or trifluoromethyl;
R² is hydrogen;
W is oxygen.

5. The nicotinanilide of the formula I according to claim 1 in which the variables are as defined below:
n is zero;
p is zero;
X² is F or chlorine;
R¹ is F or chlorine;
R² is hydrogen;
W is oxygen.

6. The nicotinanilide of the formula I according to claim 1 which is chosen from the group consisting of:
2-chloro-N-(3′,4′-dichlorobiphenyl-2-yl)nicotinamide,
2-chloro-N-(3′,5′-dichlorobiphenyl-2-yl)nicotinamide,
2-chloro-N-(3′-chloro-4′-fluorobiphenyl-2-yl)nicotinamide,
2-chloro-N-(4′-chloro-3′-fluorobiphenyl-2-yl)nicotinamide,
2-chloro-N-(3′,4′-difluorobiphenyl-2-yl)nicotinamide,
2-chloro-N-(2′,4′-dichlorobiphenyl-2-yl)nicotinamide,
2-chloro-N-(2′,5′-difluorobiphenyl-2-yl)nicotinamide,
2-chloro-N-(4′-chloro-2′-fluorobiphenyl-2-yl)nicotinamide,
2-chloro-N-(2′,4′-difluorobiphenyl-2-yl)nicotinamide,
2-chloro-N-(4-fluoro-2′,4′-dichlorobiphenyl-2-yl)nicotinamide,
2-chloro-N-(3′,4′,5′-trifluorobiphenyl-2-yl)nicotinamide,
2-chloro-N-(2′,4′,5′-trifluorobiphenyl-2-yl)nicotinamide,
2-chloro-N-(4′-chloro-2′-fluoro-5′-methoxybiphenyl-2-yl)nicotinamide,
2-trifluoromethyl-N-(3′,4′-dichlorobiphenyl-2-yl)nicotinamide,
2-trifluoromethyl-N-(4-fluoro-2′,4′-dichlorobiphenyl-2-yl)nicotinamide,
2-trifluoromethyl-N-(3′,4′,5′-trifluorobiphenyl-2-yl)nicotinamide,
2-trifluoromethyl-N-(2′,4′,5′-trifluorobiphenyl-2-yl)nicotinamide,
2-trifluoromethyl-N-(4′-chloro-2′-fluoro-5′-methoxybiphenyl-2-yl)nicotinamide,
2-trifluoromethyl-N-(3′,4′-difluorobiphenyl-2-yl)nicotinamide,
2-trifluoromethyl-N-(3′,5′-dichlorobiphenyl-2-yl)nicotinamide,
2-trifluoromethyl-N-(2′,4′-dichlorobiphenyl-2-yl)nicotinamide,
2-trifluoromethyl-N-(3′-chloro-4′-fluorobiphenyl-2-yl)nicotinamide,
2-chloro-N-(3′,4′,5′-trifluorobiphenyl-2-yl)-N-methylnicotinamide and 2-trifluoromethyl-N-(3′,4′,5′-trifluorobiphenyl-2-yl)-N-ethylnicotinamide.

7. A composition for controlling phytopathogenic harmful fungi, which composition comprises a fungicidal amount of a compound of the formula I according to claim 1 and at least one inert additive.

8. The composition according to claim 7 which can additionally comprises a further active compound.

9. A method for controlling phytopathogenic harmful fungi, which comprises treating the fungi, their habitat and/or the materials, plants, the soil or seed to be protected with a fungicidally effective amount of a compound of the formula I according to claim 1.

10. The use of a compound I according to claim 1 for controlling phytopathogenic harmful fungi.

11. Seed comprising a compound of the formula I according to claim 1 in an amount of from 1 to 1000 g/100 kg.

12. The nicotinanilide of the formula I according to claim 2 in which the variables are as defined below:
n is zero;
p is zero;
X² is F or chlorine;
R¹ is F, Cl, methyl or trifluoromethyl;
R² is hydrogen;
W is oxygen.

13. The nicotinanilide of the formula I according to claim 3 in which the variables are as defined below:
n is zero;
p is zero;
X² is F or chlorine;
R¹ is F, Cl, methyl or trifluoromethyl;
R² is hydrogen;
W is oxygen.

14. The nicotinanilide of the formula I according to claim 2 in which the variables are as defined below:
n is zero;
p is zero;
X² is F or chlorine;
R¹ is F or chlorine;
R² is hydrogen;
W is oxygen.

15. The nicotinanilide of the formula I according to claim 3 in which the variables are as defined below:
n is zero;
p is zero;
X² is F or chlorine;
R¹ is F or chlorine;
R² is hydrogen;
W is oxygen.

16. A composition for controlling phytopathogenic harmful fungi, which composition comprises a fungicidal amount of a compound of the formula I according to claim 2 and at least one inert additive.

17. A composition for controlling phytopathogenic harmful fungi, which composition comprises a fungicidal amount of a compound of the formula I according to claim 3 and at least one inert additive.

18. A composition for controlling phytopathogenic harmful fungi, which composition comprises a fungicidal amount of a compound of the formula I according to claim 4 and at least one inert additive.

19. A composition for controlling phytopathogenic harmful fungi, which composition comprises a fungicidal amount of a compound of the formula I according to claim 5 and at least one inert additive.

20. A composition for controlling phytopathogenic harmful fungi, which composition comprises a fungicidal amount of a compound of the formula I according to claim 20 and at least one inert additive.
